(19) United States
(12) Patent Application Publication
(10) Pub. No.: US 2018/0199824 A1
(43) Pub. Date: Jul. 19, 2018

(54) DEVICE FOR MEASURING BIOLOGICAL SIGNALS

(71) Applicant: BODYPORT INC., Mountain View (CA)

(72) Inventors: Corey James CENTEN, Ottawa (CA); Sarah Ann SMITH, Hamilton (CA)

(21) Appl. No.: 15/743,154

(22) PCT Filed: Nov. 2, 2015
(86) PCT No.: PCT/CA2015/051120

§ 371 Date: Jan. 9, 2018

Related U.S. Application Data
(60) Provisional application No. 62/191,318, filed on Jul. 10, 2015.

(51) Int. Cl. A61B 5/0205; A61B 5/00

(52) U.S. Cl. CPC A61B 5/0205; A61B 5/6891; A61B 5/7232; A61B 5/0402; A61B 5/7203; A61B 5/7285; A61B 2562/0252; A61B 5/0022

(57) ABSTRACT

A device for measuring a biological signal, including a cardiovascular signal. The device includes a force sensor positioned to measures a biological signal as a varying force exerted on the device and/or electrical signal sensors that cooperate with each other to measure a biological signal as an electrical signal sensed from the user. The device includes a processor that receives the biological signal, determines, from the biological signal, one or more biological parameters, and generates output representative of the one or more biological parameters.

### CROSS-REFERENCE TO RELATED APPLICATIONS

The present disclosure claims priority from U.S. provisional patent application No. 62/191,318, filed Jul. 10, 2015, the entirety of which is hereby incorporated by reference.

### FIELD

The present disclosure relates generally to methods and devices for measuring biological signals, including cardiovascular parameters. In particular, the present disclosure relates to a platform device for measuring biological signals.

### BACKGROUND

About 1 of 3 U.S. adults—or about 70 million people—have high blood pressure. Only about half (52%) of these people have their high blood pressure under control. This common condition is associated with an increase in the risk for heart disease and stroke, two of the leading causes of death for Americans.

High blood pressure is often called the “silent killer” because it typically has no warning signs or symptoms, and many people do not know they have it. Frequent monitoring of blood pressure in the home environment may be necessary to detect and keep track of blood pressure.

Most automated home blood pressure monitors employ a pneumatic cuff wrapped around the upper arm of the user. The cuff inflates to a pressure sufficient to occlude the brachial artery. Air is gradually dispelled from the cuff resulting in small blood flow oscillations that are measured and correlated to blood pressure. This technique has been employed for many years, yet many individuals at risk of or living with hypertension do not regularly measure their blood pressure. Difficulty in applying a blood pressure cuff to oneself, discomfort during the measurement process and/or lack of habit may be contributing factors to the low long-term adherence to self-monitoring.

### SUMMARY

In some examples, the present disclosure describes a device for measuring a biological signal. The device includes: a device body for supporting a user; one or more sensors for sensing at least one biological signal, the biological signal being a cardiovascular signal, the one or more sensors including at least one of: at least one force sensor positioned to sense a force exerted on the device body, wherein the at least one force sensor measures one of the at least one biological signal as a varying force; or at least two electrical signal sensors positioned to detect electrical signals from the user when the device is in use, wherein the at least two electrical signal sensors cooperate with each other to obtain one of the at least one biological signal from the user via the feet of the user; and a processor housed in the device body, the processor coupled to the one or more sensors to receive the at least one biological signal; wherein the processor is configured to: determine, from the at least one biological signal, one or more biological parameters; and generate output representative of the one or more biological parameters.

In some examples, the present disclosure describes a device for measuring a biological signal. The device includes: a device body for supporting a user; sensors for sensing respective biological signals, the biological signals including a cardiovascular signal, the sensors including: at least one force sensor positioned to sense a force exerted on the device body, wherein the at least one force sensor measures one biological signal as a varying force; and at least two electrical signal sensors positioned to detect electrical signals from the user when the device is in use, wherein the at least two electrical signal sensors cooperate with each other to obtain another biological signal from the user; and a processor housed in the device body, the processor coupled to the sensors to receive the biological signals; wherein the processor is configured to: determine, from the biological signals, one or more biological parameters; and generate output representative of the one or more biological parameters.

In some examples, the present disclosure describes a server for accessing biological information. The server includes: a processor for communication with the device disclosed herein; the processor being configured to provide the device with access to a database of stored biological information, the database including biological information obtained using the device; and the processor being configured to provide an online portal for accessing information stored in the database by an external system.

In some examples, the present disclosure describes a computer readable medium having instructions tangibly encoded thereon for execution by a processor of a computing device, the instructions, when executed, causing the computing device to: provide a user interface for at least one of providing input or receiving output from the device disclosed herein.

### BRIEF DESCRIPTION OF THE DRAWINGS

Reference will now be made, by way of example, to the accompanying drawings which show example embodiments of the present application, and in which:

FIG. 1 is a top plan view of an example of the device disclosed herein;

FIG. 2 is a bottom plan view of the example device of FIG. 1;

FIG. 3 is a view of the interior of the example device of FIG. 1;

FIG. 4 is a block diagram representing an example of the device disclosed herein;

FIG. 5 is a drawing illustrating examples of three cardiac signals that may be measured by an example of the device disclosed herein;

FIG. 6 is a block diagram illustrating communication between an example of the device disclosed herein and external system(s); and

FIG. 7 is a flowchart illustrating an example method for measuring a biological signal.

Similar reference numerals may have been used in different figures to denote similar components.

### DESCRIPTION OF EXAMPLE EMBODIMENTS

In various examples, the present disclosure describes a device, which may be in the form of a physical platform on which a user may stand, or other form for supporting a user, for measuring one or more biological signals of a user. The measured biological signal(s) may include cardiovascular parameters, such as blood pressure, heart rate, stroke volume, ejection fraction, respiration rate, ejection force, contractility, and/or pre-ejection period, for example.

In some examples, the disclosed device may be similar in size and shape to a bathroom scale and may also be capable of performing the function(s) of a bathroom scale, such as measuring the weight of the individual, estimating a body fat percentage and/or estimating a body mass index (BMI). Since many households already own a bathroom scale and most people are familiar with its use, the measurement of cardiovascular function, including blood pressure, using examples of the disclosed device may be a simple, routine task no more difficult than weighing oneself.

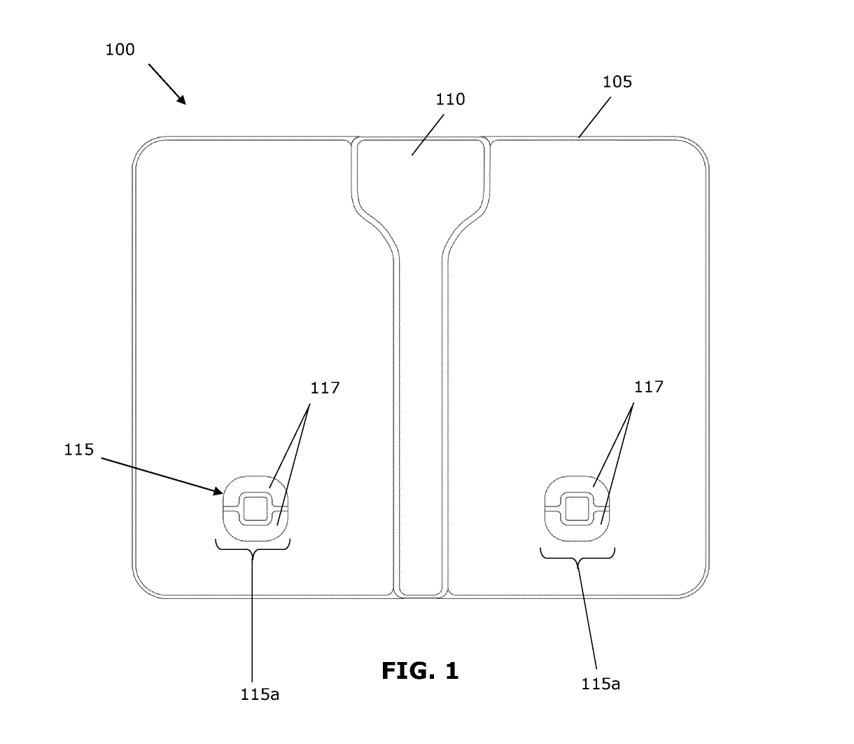

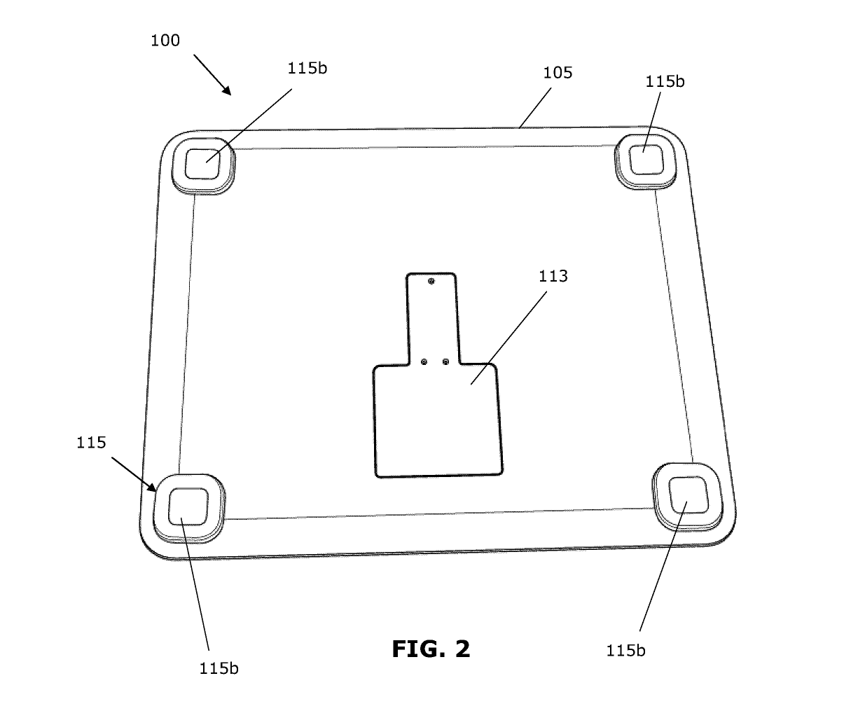

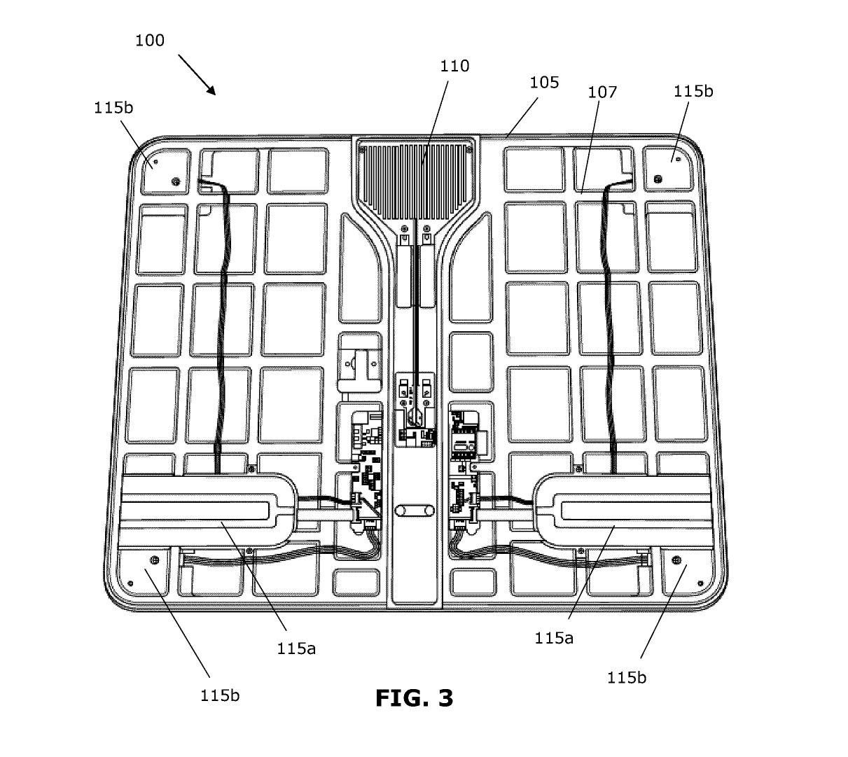

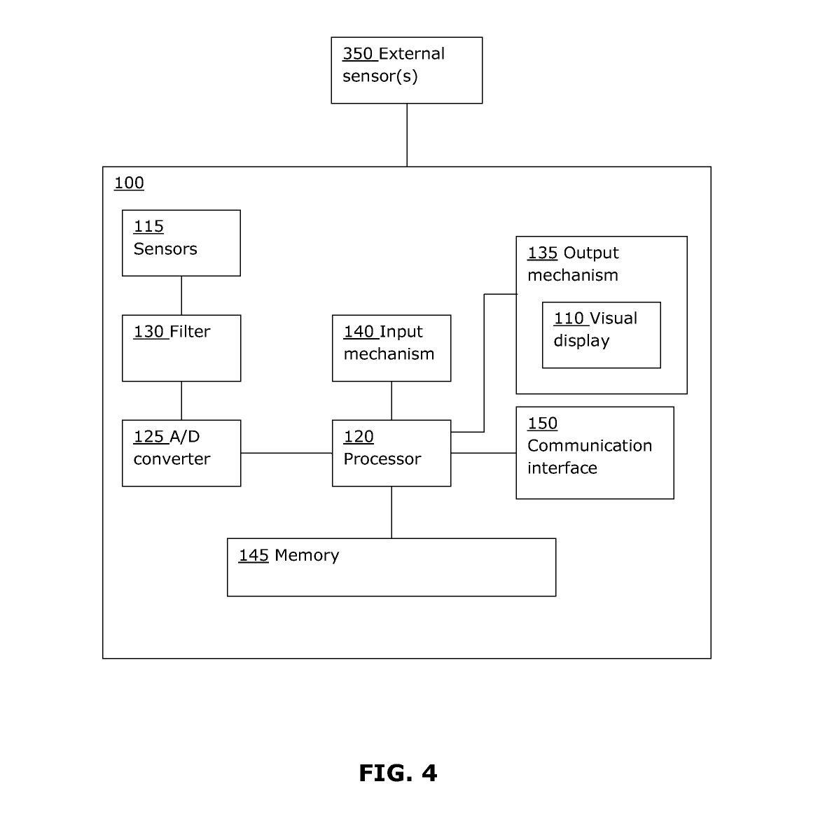

An example embodiment of the disclosed device 100 will be described with reference to FIGS. 1-4. FIG. 1 shows a top-down view of the example device 100, FIG. 2 shows a bottom view of the example device 100, FIG. 3 shows the interior of the example device 100, and FIG. 4 is a block diagram representing various components of the example device 100. In the example shown in FIG. 1, the device 100 may be configured to support the weight of a user standing on top, and may be in the form of a platform resembling a bathroom scale. The device 100 may include a body 105 that generally houses most or all of the electronic components. The device 100 may include an output mechanism, such as a visual display 110. In the example shown, the visual display 110 runs down the center of the device 100 along the centerline. The device 100 may include one or more sensors 115 for measuring a biological signal.

In the example shown, the sensor(s) 115 include two electrical signal sensors 115a, positioned on either side of the centerline approximately where the user is expected to place the heel of each foot when using the device 100. The electrical signal sensors 115a may be intended to be contact with the user (e.g., directly in contact with the user's skin or in indirect contact such as through a layer of clothing or sock), or may not require direct contact with the user. The electrical signal sensors 115a may include electrodes. For example, each electrical signal sensor 115a may include one electrode, or each electrical signal sensor 115a may include multiple electrodes 117 positioned at each heel. Generally, the electrical signal sensors 115a may operate in pairs in order to detect a signal between defined pairs of electrical signal sensors 115a, as discussed further below.

Although illustrated and described as being located to detect an electrical signal via the user's heels when the device 100 is in use, the electrical signal sensors 115a may be located elsewhere on the device body 105, for example to detect an electrical signal via the balls of the user's feet when the device 100 is in use. There may be multiple electrical signal sensors 115a located at different locations on the device body 105, for detecting signals via different portions of the user's feet, for example. In some examples, there may be multiple electrical signal sensors 115a located at each heel position. The use of multiple electrical signal sensors 115a at different locations or at similar locations may be useful for redundancy purposes, to accommodate feet of different sizes, to accommodate imprecise placement of the user's feet, for example.

The sensors 115 may also include one or more force sensors 115b. The force sensor(s) 115b may be positioned to detect the force exerted by the user on the device body 105 when the device 100 is in use. For example, the force sensor(s) 115b may be positioned on the bottom surface of the device body 105 (as shown in FIG. 2) or within the device body 105 (not shown). In FIG. 2, four force sensors 115b are provided on the bottom surface of the device body 105, each one in the vicinity of a respective corner. Such an arrangement may help to ensure an accurate and precise measurement of the user's force exerted on the device 100, including any changes in force due to cardiovascular events, as discussed below. Other configurations of the force sensor(s) 115b, including configurations using only a single force sensor 115b, may be suitable.

The device 100 may be powered by a power source such as a battery or an external power source. In the example illustrated herein, the device 100 may include a battery receptacle 113 for receiving a battery (e.g., a rechargeable battery or a standard disposable battery). In some examples, the device 100 may additionally or alternatively include a connector or port for receiving power from an external power source (e.g., a wall socket).

As shown in FIG. 3, the device body 105 may be supported by struts 107, which may be arranged in a grid pattern, to help maintain the structure of the device 100 when under the weight of a user. Each sensor 115 may be connected by wires to a power source (e.g., a rechargeable battery housed in the device 100), possibly other electrical components, and/or to a processor 120 (see FIG. 4).

The device body 105 may house the processor 120 (e.g., a microprocessor), one or more memories 145 and one or more electronic circuits. The electronic circuits may include one or more analog-to-digital (A/D) converters 125 for converting detected biological signals from the sensors 115 from an analog form to a digital form. The electronic circuits may also include filters 130 and other such circuits for removing noise and artifacts from sensed biological signals. The processor 120 may receive the processed signals from the electronic circuits. In some examples, the processor 120 may receive the unfiltered signals from the A/D converter 125 or may receive the raw signals from the sensor(s) 115, and the processor 120 may itself perform appropriate signal processing.

The memory 145 may include a database storing preset threshold(s), user information, previously acquired biological data, look-up table(s), baseline value(s), or other such information as discussed herein. The memory may also include instructions, algorithms and equations for implementing examples described herein.

The processor 120 may execute instructions stored in the memory 145 to analyze the sensed biological signals, as discussed below. The processor 120 may be coupled to one or more output mechanisms 135, for example the visual display 110, to provide output to the user. In some examples, the device 100 may optionally include one or more input mechanisms 140 (e.g., physical buttons or a touch-sensitive surface) for receiving user input. For example, the user may input control instructions to specify the biological parameter(s) to be measured. In some examples, discussed further below, the device 100 may additionally or alternatively receive user input from an external system (e.g., a user's mobile device or a desktop computing device) via a communication interface 150. Where the device 100 is in communication with an external system, the device 100 may also provide output via the external system in addition to or in place of the output mechanism 135. In some examples, the device 100 may also receive information from one or more external sensors 350, as discussed further below.

The sensor(s) 115 may be capable of measuring at least one biological signal. For example, the sensor(s) 115 may measure two or more independent signals related to cardiovascular function. Any suitable sensors for measuring biological signals may be used. For example, these sensors 115 may include piezoresistive force sensors, load cells, piezoelectric sensors, electrocardiograph (ECG) sensors, impedance plethysmography (IPG) sensors, optical photoplethysmography (PPG) sensors, magnetic field sensors or any other sensor capable of measuring a biological signal such as a cardiac or vascular signal. There may be different sets of sensors 115 provided by the device 100 for measuring different biological signals. In some examples, one set of sensors 115 may be capable of measuring more than one biological signal.

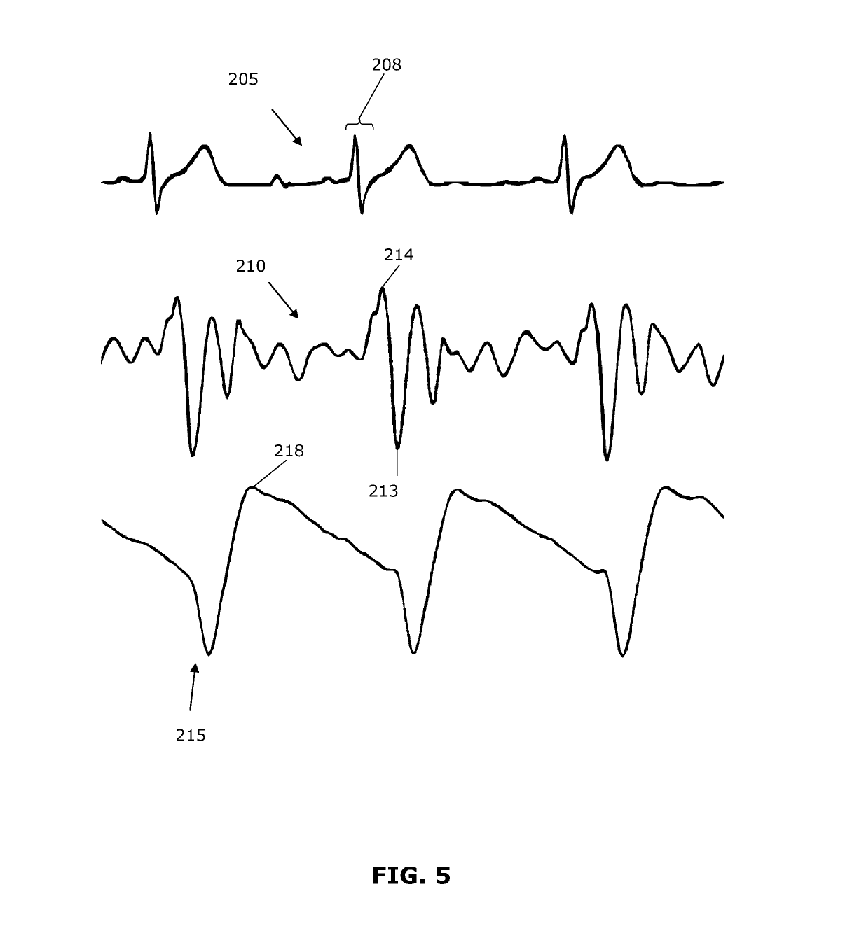

In some examples, the biological signals measured by the sensor(s) 115 may pertain to the mechanical function of the heart (e.g., ballistocardiograph (BCG)), the electrical function of the heart (e.g., ECG), and/or vascular function of the circulatory system (e.g., IPG). Examples of these three signals are illustrated in FIG. 5. FIG. 5 shows example traces of an ECG signal 205, a BCG signal 210 and an IPG signal 215. The characteristic peaks of each signal are clearly evident. The systolic time intervals (i.e. the QRS complex) may be calculated from any distinctive, repetitive feature of each signal.

For obtaining a BCG signal, the sensor(s) 115 may include one or more force sensors 115b, such as load cells, which may be positioned to detect the weight exerted on the device 100. The force sensor(s) 115b may measure the weight of the user standing on the device 100 as well as the dynamic forces exerted on the device 100 by the user. The force sensor(s) 115b and associated electronic components (e.g., analog circuitry, such as analog filters) should have a signal-to-noise ratio (SNR) and resolution sufficient to detect the small changes in the user's force exerted on the device 100 due to the ejection of blood from the heart into the aorta. There is a characteristic peak in most BCG signals known as the J wave 213 which is caused by the ejection of blood from the heart and into the aorta.

For obtaining an ECG signal, the sensors 115 may include electrical signal sensors 115a, for example including electrodes 117 positioned to detect an electrical signal via each of the user's heels when the user stands on the device 100. A pair of electrical signal sensors 115a may measure between them the potential difference generated across the body of the user by the electrical activity of the heart. ECG may be conventionally measured across the chest of a person (e.g., via contact electrodes placed across the person's chest). However, in developing the disclosed device 100, a very small ECG signal has been found to be detectable via the feet of a user. This ECG signal was found to be typically 10-100 times smaller than the typical ECG signal obtained by measurements obtained via contact at the chest or hands. Therefore, a sufficiently high resolution A/D converter 125, as well as an effective filter 130 may be appropriate to discriminate this signal. In some examples, the disclosed device 100 may use a 24 bit A/D converter, digital filtering (e.g., using the filter 130 or implemented by the processor 120) as well as an ensemble averaging technique (e.g., implemented by the processor 120) described in more detail below. The characteristic peak in the ECG is known as the QRS complex 208 and is caused by the depolarization of the right and left ventricles of the heart.

For obtaining an IPG signal, the sensors 115 may include electrical signal sensors 115a, which may be configured to apply a small, varying current to the heels of the user. In some examples, the same set of electrical signal sensors 115a may be used for sensing both the ECG signal and the IPG signal. Alternatively, different sets of electrical signal sensors 115a may be used for separately sensing the ECG and IPG signals. For example, the IPG signal may be sensed using two or four electrical signal sensors 115a (e.g., electrodes). In a configuration using four electrodes, two of the electrodes (which may be referred to as current applying electrodes) may be used to apply a current into each foot of the user, and the remaining two electrodes (which may be referred to as receiving electrodes) may be used to measure the return signal from each foot of the user. In this configuration, the receiving electrodes may also be used to measure the ECG signal.

The current may travel up the legs of the user. The pulsatile flow of blood through the user's legs presents a varying resistance to the applied current. Since blood is a conductive medium, the resistance varies with the volume of blood in the legs at any given time. The applied current encounters this change in resistivity which may cause a voltage change, detectable by the electrical signal sensors 115a, that is synchronous with the user's heartbeat. The IPG waveform has a characteristic peak 218 representative of the maximum or minimum blood volume in the legs.

The signal(s) received from the sensors, for example the BCG, ECG and IPG signals described above, may be analyzed and compared, by the processor of the device, to extract the systolic time intervals (STIs) between the occurrences of various cardiac events. These STIs may include: pre-ejection period (PEP), pulse arrival time (PAT), and/or pulse transit time (PTT), for example. PEP is related to the contractility of the heart and is known to be proportional to the time between the QRS complex of the ECG and the J wave of the BCG. Contractility, and thus PEP, is known to affect blood pressure. The PAT is the time interval between the QRS complex and the arrival of the pulse wave at the legs (peak in the IPG signal). The PTT is the time interval between the J wave of the BCG and the arrival of the pulse wave at the legs. The PAT is equal to the sum of the PEP and PTT. Since these metrics relate to the contraction force of the heart and the velocity of blood flow, they are also correlated to blood pressure.

Beyond temporal information, amplitude information may be extracted by the processor from these three example cardiac signals. Amplitude information may be used to improve the blood pressure estimate. For example, the amplitude of the J wave in the BCG is proportional to the stroke volume (i.e., the amount of blood ejected from the heart with each beat). Furthermore, the amplitude of the IPG peak is also correlated to the stroke volume since it is representative of the volume of blood in the legs with each beat of the heart. The stroke volume is also related to the blood pressure.

The measurements of the previously discussed cardiac parameters from a person who is standing in place on a surface (e.g., standing on the disclosed device) may be complicated by one or more of the following issues.

Since the example sensors described above obtain signals from the user's body via the bottom of the user's feet, certain signals may be much smaller in amplitude than when measured elsewhere on the body. For example, the ECG measured at the feet is much smaller than that measured at the chest and even the hands. The feet are located at a large distance from the heart, yet are close to each other resulting in a small potential difference. The ECG signal may be more than one hundred times smaller than that measured at the chest. Furthermore, it may be desirable for the electrical signal sensors located at the feet to be dry electrodes or capacitive sensors, to facilitate rapid use of the device. The use of dry electrodes or capacitive sensors may further reduce the amplitude of the measured signal. Very high resolution data conversion and careful analog design (e.g., via appropriate design of the electric circuitry of the disclosed device) may thus be required to accurately measure the ECG from the feet of the user. For example, a minimum A/D converter resolution of 24 bits may be used, as discussed above. Further, the common mode signal present on the dry electrodes or capacitive sensors may be measured, inverted, amplified and fed back into the user's body through an electrode that is in contact with the user's feet. This may help to reduce noise contamination from ambient sources, such as 60 Hz mains.

Any motion of a user using the device may corrupt the small signals being measured. For example, the BCG may be very small in some users and its frequency content lies in the frequency band often corrupted by sway or motion of the user standing on the device's surface. The BCG is a low frequency signal with the majority of its content between 1-15 Hz. Other signals measured by the device may also be easily corrupted by motion. ECG and IPG may experience noise due to the user changing his or her position in relation to the electrodes. This may cause large shifts in the signal amplitude that may overwhelm the signal of interest. Furthermore, motion may induce electrical artifacts, such as EMG. Excessive motion may cause large EMG spikes that interfere with the ECG signal. The EMG may be reduced with filtering (e.g., using appropriate electric circuitry in the device, or through signal processing by the processor), but may be difficult to entirely remove as it typically overlaps the frequency band of the ECG. Even in a perfectly still person, there may be EMG interference in the ECG due to the isometric muscle contractions required for balance.

The signals available at the feet typically are not as easily discernible as those collected elsewhere on the body. For example, the BCG signal contains many low frequency oscillations and peaks due to blood travelling through various vessels in the body. It is often difficult to determine the source of any particular peak in the BCG. As a result, certain signals collected at the feet may be less ideal for heart rate detection based on simple peak finding.

Certain sensor modalities may be unsuitable or impeded for sensing at the feet. For example, optical reflective sensors (e.g., for sensing PPG) may be used as an alternative to sensors for IPG. Optical reflective sensors may include an LED that illuminates the blood-filled vessels close to the surface of the skin and a photodetector that measures the light reflected back from the pulsating blood. This configuration may not be ideal for a sensor positioned below the foot; the force exerted by the weight of the body through the bottom of the foot and onto the sensor may cause the superficial blood flow to be reduced or to cease. While certain mitigations are possible, such as careful positioning of the sensor or deeper light penetration by selection of light wavelength and sensor separation distances, the implementation of this type of sensor may not be ideal. In examples of the disclosed device, an IPG sensor (e.g., an electrical signal sensor that sends a current through the user, such as described above) may be used rather than an optical sensor due to the accuracy of the IPG sensor being independent of positioning and contact force. These characteristics of the IPG sensor may make it more suitable for measurements when positioned under the foot.

In various examples, the disclosed device may implement various solutions to overcome one or more of the aforementioned sensor issues. For example, the disclosed device may use a suitable gating signal to act as a reference for all other measured signals. This gating may be implemented by the processor of the device, or through the use of timing circuitry. The selected gating signal should have a sufficiently high SNR such that characteristic signal features may be easily extracted. For example, the IPG signal may be chosen as the gating signal. The IPG is a relatively large amplitude signal resulting from blood flow in the legs of user. Furthermore, as a modulated applied current at a specific frequency, the IPG signal has been found to be relatively immune to ambient noise. The modulated frequency may be chosen to be between 10 kHz and 100 kHz. For example, the modulation frequency may be selected to be 64 kHz. This frequency is above that of common noise sources such as mains electricity at 60 Hz. In addition, the IPG signal may be more consistent in its amplitude and morphology compared to other biological signals, such as the PPG. The IPG is a signal originating from blood flow in the large vessels of the lower torso and legs and may not be seriously impacted by contact forces, ambient temperature or other external factors.

Once a suitable gating signal is selected, the gating signal may be filtered (e.g., using appropriate signal processing by the processor and/or using appropriate electrical circuitry in the device) to isolate the gating trigger, in the case of the IPG signal the pulsatile component due to blood flow. The filtering may be performed by an analog filter applied prior to digitization by the A/D converter or by a digital filter implemented by the processor, for example. In the case of the IPG signal, the pulsatile component is part of a much larger DC component due to the impedance of the user's body.

For sensed cardiovascular signals, the selected gating signal should contain evident periodic peaks synchronized with the user's heartbeat. These peaks may be detected with a peak detection algorithm implemented by the processor. The time at which each peak occurs may be used to align the sensed cardiac signals. The timing reference may also be derived from other features on the waveform. For example, the lowest point of the IPG wave may be chosen as a reference instead of the peak, since the lowest point may be less corrupted by wave reflections than the maximum peak. This may be due to the fact that the lowest point in the IPG signal occurs during diastole. The peak of the first derivative of the IPG signal may also be used as the timing reference. This point denotes the maximum slope of the rise in the IPG signal. This may be a suitable reference point due to its consistency and ease of detection. Other methods, such as a tangent intercept may also be used.

An ensemble average may then be performed over multiple heartbeats for each cardiac signal. Ensemble averaging of the cardiac signals over multiple heartbeats may help to reduce or eliminate noise and expose detailed features of the signal that may otherwise be obscured. The ensemble averaging technique may be effective at reducing EMG contamination in the ECG signal while exposing the QRS complex. It may also be effective at eliminating the amplitude modulating effects of respiration on the ECG and BCG signals, thereby making their amplitudes more repeatable and clinically useful. Particularly noisy signals may be ensemble averaged over more heart beats to further reduce contamination, for example.

The number of heartbeats over which a signal is to be averaged may be dynamically adjusted by the processor. The processor may evaluate the SNR and/or standard deviation of the cardiac signals, to determine the number of heartbeats over which the signal should be averaged. For example, the processor may extend the averaging window for a signal until the ensemble averaged signal is below a predetermined threshold for SNR and/or standard deviation. Therefore, the duration of a measurement may be impacted by the quality of the signals. If the processor determines that the signal quality of one or more sensed signal is poor, an accurate blood pressure measurement may require data collected over a greater number of heart beats (e.g., thirty heart beats) compared to a lower number of heart beats (e.g., ten heart beats) for a higher quality signal.

The user may be provided with instructions or indication, via the output mechanism, to instruct the user to remain in position for the necessary length of time to obtain a sufficient measurement. For example, the visual display may provide graphical or textual instructions for the user to keep both feet in position over the sensors for the necessary length of time. Alternatively, or additionally, the device may include a light that flashes or changes colour when the necessary length of time has elapsed, or the device may provide audio or tactile cues when the necessary length of time has elapsed. Failure of the user to remain in position over the sensors for the necessary length of time may result in the output mechanism indicating an error (e.g., display of an error message on the visual display).

The device may also be configured to detect bad peaks or false peaks in the gating signal to prevent the addition of corrupt or noisy data to the ensemble averages. For example, peaks in the gating signal due to excessive user motion or poor electrode contact (e.g., where the sensors include contact electrodes) may be ignored. Detection of excessive motion may be implemented by monitoring the BCG or force signals using the device's force sensor(s). Large movements or swaying by the user will tend to result in large deviations from a baseline weight. A baseline weight value may be calculated using a low pass filter or moving average. The current instantaneous weight measurement may be compared to the baseline weight measurement to detect large fluctuations related to motion. If motion is detected, any peaks in the gating signal occurring during a window of time around the movement may be ignored. Similarly, contact with the IPG and ECG electrodes may be measured by examining the baseline impedance value across the electrodes and ignoring peaks in the gating signal when any large fluctuations are detected.

Once the ensemble averages of all three cardiac signals are calculated, the relevant cardiac parameters may be calculated. As previously discussed, STIs may be correlated to various cardiac parameters. These intervals may be calculated using the ensemble averages of the cardiac signals. The time intervals may be calculated as the time differences between the peak amplitudes in each of the three cardiac signals. As previously discussed, these peak values may correspond to a specific point in the cardiac cycle. For example, the peak in the ECG may correspond to the QRS complex which denotes the electrical depolarization of the heart. Alternatively, the systolic time intervals may be calculated using cross-correlation of the ensemble averaged signals. For example, the maximum value of any two cross-correlated signals may represent the time delay between the peaks of each signal. The time intervals may also be calculated as the time between other repeating features of the waveform (e.g., the time between minimums, between the peaks of the first or second derivatives, etc.).

The features in the waveform used to calculate the time intervals do not have to be maximums or peaks. They may be other features of the waveform, such as the minimum, the foot or the peak 1^{st} or 2^{nd} derivatives.

Once the STIs are determined, blood pressure and other cardiovascular metrics may be calculated. One such time interval, PTT, is significantly correlated to blood pressure. It is also inversely proportional to pulse wave velocity (PWV). Pulse wave velocity is a reliable measure of arterial stiffness. PWV is an independent predictor of all-cause mortality and many adverse cardiovascular events. It is a metric considered valuable in the treatment and diagnosis of hypertension. In some examples, the device may measure PTT and/or PWV and may communicate these values to the user and/or a physician. Furthermore, if the height of user is known (e.g., entered into the device using an input mechanism, determined from an internal or external database, or communicated to the device from an external system), PWV may be calculated from PTT since the vessel length over which the pulse wave travels is correlated to height. For example, the PTT may be calculated as the time difference between the peak amplitude in the BCG wave and the peak amplitude in the IPG wave. In this case, the PTT represents the travel time of the pulse wave from the aortic arch to the user's legs (iliac or femoral artery). This distance is correlated to height.

The PTT may also be calculated from one signal. For example, the BCG signal represents the flow of blood up the ascending aorta (denoted as the I wave, 214 in FIG. 5) and down the descending aorta (denoted as the J wave, 213 in FIG. 5). The time difference between the I wave and J wave may be indicative of the PTT of the blood flowing through the aorta. In this example configuration, no other signal is required to obtain the PTT value. In this example, a second cardiac signal that has a higher SNR may be obtained to act as a gating signal for performing ensemble averaging on the BCG signal. This may help to improve the SNR of the BCG features for more accurate measurement and calculation of the required time intervals.

PTT nd PWV may also be used to calculate a user's blood pressure. The Moens-Korteweg equation is often employed in blood pressure calculations, describing the relation between blood pressure and pulse wave velocity. It assumes that the PWV in a short elastic vessel is obtained from its geometric and elastic properties and given by:

$$PWV = \frac{distance}{PTT} = \frac{distance}{\sqrt{\frac{E \cdot h}{2 rp}}} \qquad ( 1 )$$

E relates to the elasticity modulus of the vessel wall, h is its thickness, p is the density of blood, and r is the radius of the vessel. Blood pressure and PWV are interconnected by the relation of elasticity and blood pressure in Hughes equation:

E=E e^{αP} (2)

where α≈0.017 mmHg−1. Pressure, P, in this case is the mean arterial pressure (MAP). Based on these equations, calibration functions may be derived to translate PTT to blood pressure assuming constant vessel thickness and radius. By combining both equations, a logarithmic dependency is found:

P=A·ln PTT+B (3)

where P is either systolic blood pressure, diastolic blood pressure or mean arterial pressure, depending on the coefficients chosen for A and B. In some examples, this pressure, P, may be correlated to the user's diastolic blood pressure. The systolic blood pressure may be found by adding a term to the diastolic blood pressure that is proportional to stroke volume. The stroke volume measurement may be derived from the amplitude of the BCG signal or the amplitude of the IPG signal. Alternatively, the stroke volume may be calculated using the amplitude of the IPG signal.

The calculation of stroke volume may rely on a number of variables, for example including heart rate and weight. Since stroke volume is typically correlated to the size of a person, the example disclosed device may be convenient in that weight and body surface area (BSA) may be easily measured with little or no additional user input. A stroke volume calculation may take the form of:

SV=R+S·HR+T·BSA+U·Age+V·ET (4)

where SV is the stroke volume, HR is heart rate, BSA is body surface area (which may be calculated from height and weight) and ET is ejection time (which may be derived from the BCG or IPG signal). R, S, T, U and V are constants derived from personal or universal calibrations.

Other equations may be used for the calculation of blood pressure. For example, if it is assumed that collagen recruitment has initiated, equation (3) may be simplified to an inverse relationship between P and PTT:

$$P = \frac{A}{PTT} + {K}_{2} \qquad ( 5 )$$

Examples of the present disclosure may enable measuring of multiple time intervals. For example, when ECG, IPG and BCG are simultaneously collected, PTT, PAT and PEP may be measured concurrently. This may allow different time intervals to be used in equations (3), (4), and (5). For example, PAT may be used instead of PTT. Since PAT includes PEP, it may be more representative of the contractility of the heart itself than is PTT. In some examples, the systolic and diastolic blood pressures may be calculated using the PAT and PTT values respectively. For example:

SBP=A·ln PAT+B (6)

DBP=C·ln PTT+D (7)

where SBP and DBP are systolic and diastolic blood pressure, respectively.

The A, B, C and D parameters may be derived using manual or automated calibration procedures. For example, during a calibration phase, a cuff-based blood pressure monitor may be worn by the user while using (e.g., standing on) the device. The cuff may be configured to inflate while a measurement is taken by the device. The cuff-based measurement may be used to derive the A, B, C and D coefficients in the above equations. To improve the accuracy of the calibration, multiple blood pressure measurements may be taken with the cuff while the user is using the device. The cuff measurements may be automatically transmitted (e.g., via wired or wireless communication) to the device for calibration or may be manually input by the user. The output mechanism of the device may provide instructions to the user to carry out the appropriate calibration steps, for example through textual or audio prompts.

In some examples, the device may not require individual person-to-person calibration. For example, when the device has access to sufficient data across the general population (e.g., using data collected by the device itself, or accessed from an external database), the device may be able to determine the calibration coefficients based on characteristics of a specific user. In some examples, the device may store or access a look-up table for determining the calibration coefficients based on specific user information. This user information may be manually input by the user (e.g., using a computer or smartphone in wired or wireless communication with the device, or via an input mechanism on the device itself). In some examples, this user information may be automatically gathered by the device. For example, the information about the user that may be relevant in calculating calibration parameters may include weight, height, age, gender, smoking habits, genetic information, family history, blood test results, cholesterol levels and/or existing diseases, among others. Where the device is in the form of a platform on which the user stands, one or more of these parameters may be conveniently collected automatically. For example, the user's weight may be measured automatically (e.g., using force sensors in the device) along with blood pressure measurements, allowing calibration coefficients to be adapted to a person's body as it changes in weight.

Other metrics measured by the device may be used to enhance the accuracy of the coefficients in equations (3), (4), (5), (6) and (7). For example, the amplitude of the BCG signal may be correlated to stroke volume. Stroke volume may be used to calibrate the systolic blood pressure. In particular, the BCG may be representative of the force of contraction with each heartbeat. This data may be correlated to blood pressure. Furthermore, the force of contraction may be compared to the user's body weight. A ratio of contraction force to body weight may be calculated by the processor to provide an additional metric by which the blood pressure may be calibrated. Furthermore, the device may derive pre-ejection period (PEP) from the cardiac signals. Multiple studies have demonstrated a relationship between PEP and blood pressure since PEP is directly related to the contractility of the heart muscle. PEP may also be used to enhance the accuracy of the blood pressure calibration.

The various metrics, whether measured by the sensors or inputted by other means (e.g., manually by the user or from an external system), may be used to classify an individual on a certain blood pressure curve over which their blood pressure is directly proportional to ln(PTT). The metrics may also be used to train the device, using machine learning or other classification algorithms, to accurately estimate the user's blood pressure.

Beyond blood pressure, other cardiovascular metrics may be calculated by examples of the disclosed device. Ejection fraction is the amount of blood pumped by the heart as a ratio of the blood in the heart prior to contraction. Ejection fraction is correlated to a ratio of pre-ejection period (PEP) and left-ventricular ejection time (LVET). PEP may be found using the ECG and BCG correlation. The LVET corresponds to the duration of ejection of blood from the heart during a contraction. LVET may be found through contour analysis of the BCG or IPG signal. The duration of certain features in the BCG and IPG correspond to LVET. Therefore, these quantities may be combined to calculate the PEP/LVET ratio. Ejection fraction is a critical parameter in diagnosing and monitoring heart failure. An ejection fraction below 55% is often indicative of heart failure. Therefore, careful monitoring of changes in the PEP/LVET ratio may help detect a worsening of heart failure. This metric may also be an early indicator of a decompensation event. By detecting such an event well before it happens, hospitalization may be avoided through proper drug titration.

Other metrics such as heart rate and heart rate variability may be easily extracted from the cardiac signals obtained by the device. While only one cardiac signal may be required to calculate these parameters, the calculation may be more robust by calculating them multiple times across each of the three signals and averaging the result. For example, heart rate variability is correlated with certain heart conditions such as atrial fibrillation. It may also be a measure of stress levels. This value is measured as the standard deviation of time delays between consecutive heart beats. This metric may be independently measured on the ECG, BCG and IPG signals. The HRV measurements corresponding to each signal may be compared and averaged for increased accuracy.

Metrics representative of biological function other than heart health may also be measured. For example, the cardiac signals may be modulated by the user's breathing. In particular, the BCG may be sensitive to the body movements associated with low frequency inhalation and exhalation. By low pass filtering (e.g., using appropriate filter circuitry or via signal processing by the processor) the BCG signal to below 1 Hz, these breathing motions may be extracted to calculate a resting respiratory rate of the user. Furthermore, the amplitude of the respiratory signal may be used to estimate the expiratory volume of each breath. This signal may be correlated to parameters normally obtained using a spirometer such as forced expiratory volume (FEV).

In some examples, measurements related to the user's body (e.g., weight, body fat and/or height), which may be automatically obtained by the device and/or manually entered by the user, as discussed herein, may be used to calculate a cardiovascular metric, in addition to or in place of being used to perform a calibration. For example, cardiac output may use height and weight information for calculating BSA, which in turn may be used to calculate a cardiac index. A cardiac index value that may be considered normal or healthy for a user of a certain body type or size may be considered abnormal or unhealthy for a user of a different body type or size. Thus, by enabling the ability to compare calculated metrics to body characteristics, examples of the disclosed device may provide further utility.

Beyond cardiac signals, the device may determine and optionally record other parameters that may be useful in calculating an accurate blood pressure. For example, the user's anatomy and physiology may affect the correlation coefficients between certain cardiac parameters and blood pressure. For example, the systolic blood pressure (SBP) may be calculated taking into account the user's age, height and gender as follows:

$$SBP = A \cdot age + B \cdot height + C \cdot gender + \frac{D}{PTT} + E \cdot SV \qquad ( 8 )$$

where gender has the value 0 or 1 for male or female, respectively.

In some cases, the weight and height of the user may be useful information. For example, the blood pressure may be derived from a look-up table that retrieves parameter values from the table based on user characteristics and/or calculated biological parameters (e.g., age and PTT), with the parameter value to be used in equations such as:

SBP=(r1,c1)·PAT+(r2,c2)·weight (9)

DBP=(r3,c3)·PTT+(r4,c4)·weight (10)

where (rx,cx) is the retrieved parameter value from the look-up table.

As noted above, where the device is in the form of a platform or other configuration that supports the user's entire weight, the device may be convenient for assessing the weight of the user. For example, the user's weight may be detected using the same force sensors employed for the measurement of other biological signals, such as the BCG signal.

For adults, height rarely changes and the user may be prompted to provide this value only for a first-time use, such as during an initial calibration phase. For example, the height value may be input using a software user interface provided on a mobile computing device or other desktop computing device, which may in turn communicate (e.g., via wired or wireless communication) the value to the device. The height may also be inputted using the input mechanism on the device, such as using integrated buttons and a display provided on the device. In some examples, a height measurement may be obtained without the user entering the height value explicitly. For example, a user interface on a mobile computing device may prompt the user to hold the mobile device at chest or heart height while standing on the disclosed device, with the mobile device's camera facing downwards toward the disclosed device. The mobile device may capture an image of the disclosed device and use the spacing between known visual features of the disclosed device to calculate the height of the mobile device above the disclosed device. For example, if the disclosed device includes a display, the display may show a specific pattern that may be used by the mobile device's camera to determine its height above the disclosed device. Other sensors within the mobile device may also be used to calculate its height, such as an altimeter. Other devices, such as a wearable fitness bracelet or smart watch may also be used to determine the user's height in a similar manner. In some examples, the disclosed device may include or communicate with a height measuring tool, for example a vertical height measure, similar to that found in a physician's office, which may electronically communicate the measured height to the device's processor.

In some examples, the sensors of the disclosed device may include electrodes for measuring an IPG signal, that may be further used for measuring the body fat of the user. Similar to obtaining an IPG signal, for example as discussed above, a body fat measurement may be obtained by applying a current to the user and sensing an impedance. The AC portion of the sensed impedance may be detected as the IPG signal, while the baseline DC impedance may be correlated to the patient's body fat. Appropriate algorithms may be used by the processor to convert the detected impedance value to a body fat value. This body fat information may be used to more accurately calculate blood pressure by accounting for variations in the cardiac signals due to body fat, for example. The proportions of adipose and lean tissue may affect the shape of the BCG signal by dampening the mechanical signal to different degrees. This dampening effect may be compensated using body fat and weight information. The measured body fat information may also be a measured biological parameter that may be outputted and/or stored.

In some examples, the device may also include or receive information from sensors capable of providing contextual information about the biological measurements obtained. For example, the device may include or receive information from temperature and humidity sensors to provide information that may be used by the processor to determine the effect of ambient conditions (e.g., temperature, pressure and/or humidity) on the user's blood pressure. Furthermore, microphones, light sensors, pressure sensors and/or other context sensors may provide information to enable the processor to track the impact of environment and weather on an individual's heart health. Where the device stores measurements and information about the user (e.g., to an internal or external database), such stored information may be stored in association with information about the context in which the measurements were obtained. The context sensors may also provide information that may be used to improve the accuracy of the measurements. For example, temperature and humidity measurements obtained by temperature and humidity sensors (which may be internal or external to the disclosed device) may be used by the processor to perform compensation on raw data that may be affected by environment. For example, electrodes (e.g., for measuring ECG and IPG) may have a lower resistivity at a higher humidity. As another example, the gain of analog circuitry in the device might deviate as temperature fluctuates, and the processor may be able to compensate for this using information obtained from context sensors.

In some examples, the device may also measure the posture and/or balance of the user. For example, using force sensors positioned in the user-supporting device body (e.g., four load cells positioned in the corners of the device body), a center of pressure of the user standing on the device may be calculated. The amount of deviation or variance in the center of pressure during the course of a measurement may be used to detect symptoms of high or low blood pressure. For example, an individual with low blood pressure upon standing may experience dizziness and/or a poor sense of balance. The device may detect a deviation or variance in the user's center of pressure that is greater than a predetermined threshold, and accordingly determine that the user is exhibiting dizziness and/or poor balance, which may be indicative of low blood pressure. The device may generate an output to alert the user and/or a physician accordingly. The user's balance and sway may also be used to detect the influence of certain blood pressure medications over time. For example, certain blood pressure medications may induce dizziness and/or postural instability that may be measured by the disclosed device, as described above. By monitoring these effects, medications may be more accurately titrated for a user by their physician using the collected data.

In some examples, the device may be used to predict and/or prevent adverse cardiovascular events. By determining parameters (e.g., blood pressure) relevant to myocardial infarction, cardiac arrest and stroke, deviations from normal signals (e.g., blood pressure values expected of a similar healthy person) may be detected and a warning may be outputted to the user and/or a physician. For example, abnormalities in the ECG signal may be indicative of certain congenital or acquired heart defects. The processor may compare the measured ECG signal against an expected ECG pattern for a healthy person and may determine that there is a significant abnormality if the measured signal deviates from the expected pattern by more than a predefined amount (e.g., missing certain expected peaks in the measured ECG signal). Disturbances or artifacts in the BCG signal, which may be detected by the processor in a similar way, may precede a heart attack as such disturbances may indicate changes in the mechanical functioning of the heart. These changes may be caused by weakness in the cardiac muscle, for example. Irregularities in vascular signals, such as the IPG signal, may be similarly detected, and the presence of such irregularities may be determined to be a possible problem in the circulatory system. The IPG signal represents blood flow in the iliac and femoral arteries of the legs. Peripheral arterial disease, deep vein thrombosis or other circulatory issues may cause abnormal measurements.

The measured biological signal may be compared by the processor against a predefined expected value or pattern for a similar (e.g., similar age and height) healthy person. In some examples, the measured biological signal may be compared to the user's own baseline value or pattern, which may be calculated by the device and stored as the user's “normal” or “healthy” state. A baseline measurement may be obtained by performing a long-term ensemble average of measurements for each signal. The long-term ensemble average may be calculated by averaging, over a number of days, the daily average for a given signal. This baseline may be cumulative and continuously updated. For example, each time a new measurement is taken, the newly collected signals may be added to the long-term ensemble average.

Each time a measurement is taken, the processor may compare the new measurement to the baseline for the user. If the processor determines that the new measurement is significantly different from the baseline (e.g., differs by an amount greater than a predetermined threshold or differs by more than three standard deviations), this may be indicative of an upcoming or current adverse event. The new measurement may not be added to the baseline. The processor may flag the new measurement as a possible measurement error and/or adverse event. In some examples, the processor may compare a current measurement against a preset threshold (e.g., predefined according to medical guidelines for a healthy person) regardless of the user's baseline measurement, and determine an upcoming or current adverse event if the current measurement exceeds the threshold. The preset threshold may be set according to the user's characteristics (e.g., age, sex, height, etc.). The preset threshold(s) and/or user-specific baseline referenced by the processor may be stored in the device's own internal memory, or may be accessed from an external database.

When the processor determines that one or more measurements are indicative of an upcoming or current adverse event, the processor may cause the device to output appropriate warning to the user (e.g., via an audio or visual output) and/or communicate the possibility of an adverse event to a physician (e.g., via wired or wireless communication to an external physician workstation). The communication may include details of the measurement(s) that caused the determination of the adverse event and may include one or more suggestions for the user to reduce the chance of the adverse event occurring.

In some examples, the device may be capable of distinguishing between individual users. This user identification may be used to automatically upload the measured data to the correct user profile (e.g., stored in an internal or external database) when multiple individuals are using the same device.

For example, weight and body fat measurements may be used to discriminate between different users. The weight and impedance values typically fluctuate slowly over time and differ between individuals. For example, the device may access an internal or external database of user identification, where each user identifier is associated with the user's information including, for example, baseline weight and body fat measurements. The database may also contain information other information discussed herein, such as user characteristics (e.g., height, age, sex, etc.), baseline biological measurements and/or a record of previous measurements, in association with the user identifier. When a user uses the device, the processor may compare the obtained weight and body fat measurements to the values in the database to determine the appropriate user identifier (e.g., the user identifier having a similar stored associated weight and body fat baseline). If a match is found, the device may cause all measurements obtained in this session to be associated with the identified user identifier. Optionally, the user may be prompted to confirm that the user identifier is correct. If no match is found, the device may determine that this is a new user and may generate a new user identifier or prompt the user to enter a new user identifier.

To add more specificity to the identification of the user, cardiac signals may also be used to determine a certain user. For example, the ECG and BCG signals are known to exhibit features that are unique to each individual. These signals may be used as a cardiac signature, similarly to use of weight and body fat described above, for user identity determination in addition or as alternatives to use of weight and impedance measurements.

In some examples, the device may be capable of wired or wireless communication with external systems, for example using internet communication. Communications between the device and any external systems may be encrypted for privacy and security, for example. The device may include one or more wireless communication interfaces, for example.

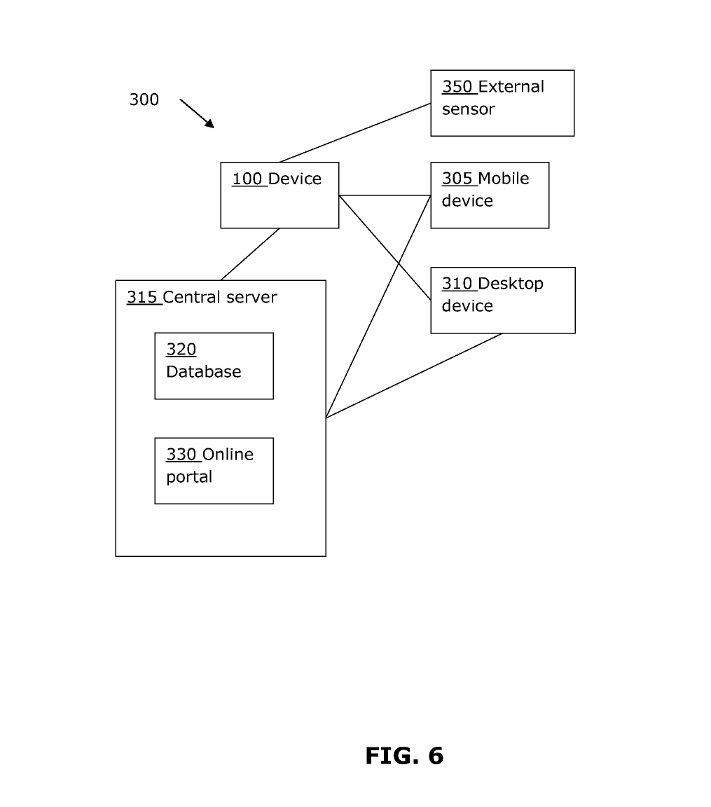

FIG. 6 is a block diagram illustrating an example system 300 including the disclosed device 100. In the example system 300, the device 100 may communicate with one or more mobile devices 305 (e.g., cellular telephone, smartphone, personal digital assistant, laptop computer, etc.), one or more desktop devices 310 (e.g., personal computer, workstation, etc.) and at least one central server 315. As discussed previously, the device 100 may also communicate with one or more external sensors 350. The device 100 may receive sensed data (e.g., environmental data) from the external sensor(s) 350, for example as described above.

The device 100 may interface with the user via the mobile device(s) 305 and/or desktop device(s) 310. For example, the device 100 may be used with a proprietary software application executed by the mobile device(s) 305 and/or desktop device(s) 310, to provide a user interface. The user or other authorized person (e.g., physician or family member) may provide input (e.g., user information and/or control instructions) to the device 100 and may receive output from the device 100 (e.g., output of calculated biological parameters, user prompts and/or warnings) via the user interface. The user interface may be intended to be used while the user is using the device 100 (e.g., to assist in calibrating the device 100, such as discussed above). Such a user interface may be used in place of or in addition to any output/input mechanisms provided on the device 100 itself.

The device 100 may communicate with the central server 315 (e.g., over an internet connection or over a private network) to upload data to and/or access data from an external database 320 maintained by the central server 315. For example, the measured signals from the device 100 may be automatically uploaded (e.g., at preset time intervals or after every measurement session) to the central server 315 to be stored in the database 320. The central server 315 may have in place security mechanisms (e.g., encryption) to ensure that the data in the database 320 is kept secure and private. In some examples, the database 320 may be a cloud database, and may be stored over several servers.

The central server 315 may also provide an online portal 330 that may be accessible via the mobile device(s) 305 and desktop device(s) 310, to provide access to information stored in the database 320. This online portal 330 may provide secure access to the secure repository of a user's health data in the database 320. For example, the online portal 330 may enable a user or other authorized person (e.g., the user's physician or trusted family member) to review trending health information, real-time biomedical signals and/or other relevant information for the user. A physician may access the user's data to help diagnose disease, prescribe medications or monitor treatment progress. A family member may monitor the well-being of the user to ensure a specific health condition does not progress without warning.

Internet connectivity may enable the device 100 to obtain information from other external sources to enhance its accuracy and/or predictive capabilities. For example, the device may compare ECG data to an online database of ECG signals collected from both healthy and unhealthy individuals (e.g., collected using the device 100 or collected by other means). For example, the device 100 may be able to access anonymized data stored in the database 320, which may include a collection of anonymized measurements from possibly plurality of devices 100 being used by different users. The database 320 may be regularly updated with new data (e.g., uploaded by a plurality of devices 100), which may provide information to help improve detection accuracy of each device 100. In some examples, the device 100 may communicate with an externally maintained database (e.g., one or more electronic health records maintained by an institution) for similar purposes.

In some examples, the device may interact with other external systems to enhance its data. For example, a separate wearable device capable of measuring activity levels may be used to provide data to supplement the weight and blood pressure data obtained by the disclosed device. Increased activity levels may correlate to decreases in weight and blood pressure. This data may provide further actionable insight into the cause of high or low blood pressure. Furthermore, the activity data may be used to determine the state of the user prior to taking a measurement. For example, if the person was running prior to the measurement, heart rate and blood pressure may be elevated relative to the same measurement taken immediately after an extended period of rest. This contextual information may help a physician in discriminating between normal blood pressure values and those artificially elevated by external factors. Such contextual information may also be used by the device to avoid triggering incorrect warnings of possible adverse events and/or may enable the device to compare the obtained measurements against a different baseline (e.g., a baseline for post-exercise heart rate rather than a baseline for resting heart rate).

A change in measured heart rate may be used as an indicator of activity prior to measurement. For example, if the user stood up from a seated position immediately prior to the measurement, the user's heart rate may be elevated but will quickly fall during the course of the measurement. This fall in heart rate may be detected by the device and used to categorize the measurement differently and/or may be used to extend the measurement period until the heart rate has fully stabilized to ensure accuracy.

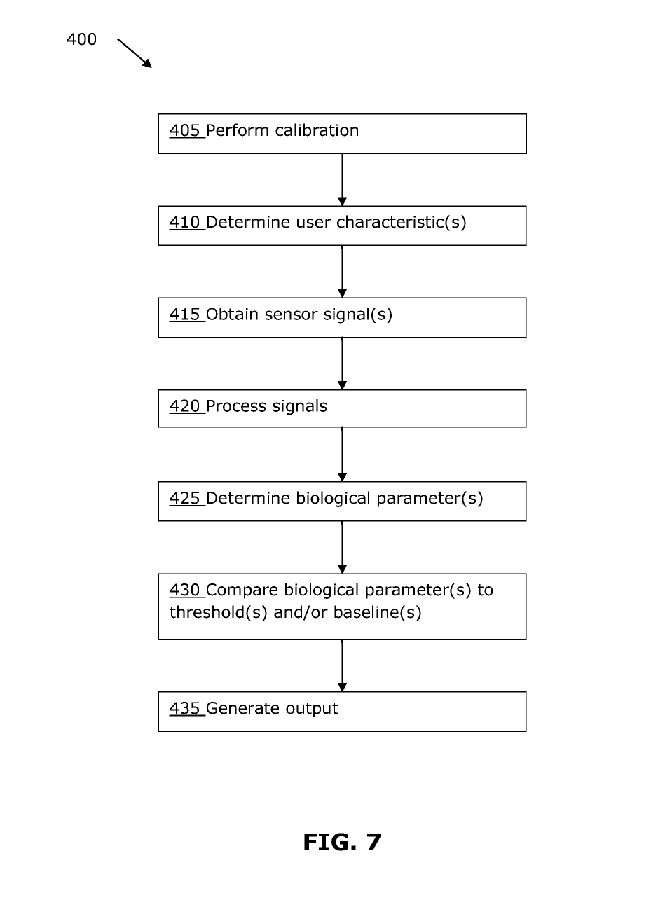

FIG. 7 is a flowchart illustrating an example method 400 that may be performed by the processor to measure a user's biological function and provide appropriate output. The method 400 is a general example of how the present disclosure may be implemented, however one or more steps described below may be omitted or switched in order.

At 405, the processor may perform calibration. For example, calibration may be performed to determine the user's height and other user characteristics (e.g., age, sex, etc.). This calibration may be performed only for a first-time user. In some examples, calibration may be performed at set intervals (e.g., yearly) to ensure the user characteristics are up-to-date.

At 410, the user characteristic(s) (e.g., age, height, sex, etc.) may be determined. For example, the processor may retrieve previously stored information from an internal or external database. In some examples, the processor may cause output to be provided to the user (e.g., via the device's output mechanism or via an external device) to prompt the user to input user characteristic(s).

In some examples, 405 and/or 410 may be omitted where such information is not used (e.g., where biological function is compared to a baseline without having to know the user's age, height, sex, etc.).

At 415, the processor receives sensed signal(s) from the device sensors and/or other external sensor(s). The received signal(s) may be processed or partially processed, for example by other circuitry (e.g., A/D converter and/or filter components) in the device. In some examples, the received signal(s) may include context information (e.g., environmental data such as temperature and humidity) as well as sensed biological signal(s) (e.g., ECG, IPG and BCG signals).

At 420, the processor may perform signal processing. This may include, for example, performing digital filtering (in additional to or in place of filtering by an analog filter), gating (e.g., using an appropriate gating signal) and ensemble averaging. The signal processing may be performed to reduce or remove noise and other artifacts from the received signal(s).

At 425, the processor may calculate one or more biological parameters (e.g., blood pressure, body fat, BMI, etc.) using the processed signals. Such calculations may include the use of a look-up table (e.g., to determine appropriate coefficient constants) and/or information about user characteristics and/or ambient conditions, for example.

At 430, the processor may compare the calculated biological parameter(s) to preset threshold(s) and/or predetermined baseline value(s). Based on this comparison, the processor may determine whether an adverse event is expected, for example.

At 435, output may be generated. For example, the processor may cause the output mechanism(s) of the device to provide output to the user representing the calculated biological parameter(s) and/or based on the determination of whether an adverse event is expected. The output may additionally or alternatively be provided to one or more external systems (e.g., a mobile device or a workstation), for example via a user interface. The output may additionally or alternatively be provided to an external database (e.g., managed by a central server) to maintain a record of the user's health history for example.

Various examples of the disclosed device may be constructed using various materials. For example, the electrodes for sensing ECG and IPG signals may be constructed of any suitable conductive material. Stainless steel may be suitable due to its high conductivity and corrosion resistance. Conductive polymers or other metals may also be used. Conductive sputtered substrates may also be used to form the electrodes. For example, indium tin oxide sputtered glass or plastic may form the sensors areas.

In some examples, the electrical signal sensors may be capacitive sensors. Capacitive sensors may require no direct physical contact between the skin of the patient and the device. The cardiac signals may be capacitively coupled from the body using a very high impedance front end. This configuration may enable the device to obtain signals without direct contact with the user's skin, for example from a user wearing socks. Both ECG and IPG signals may be obtained using capacitive sensors. In the case of IPG, both the applied and received signals may be coupled capcitively to the user, for example. These capacitive sensors may be more sensitive to ambient noise and may have a diminished SNR.

In some examples, the device may include multiple force sensors (e.g., load cells) to measure the weight and BCG. For example, there may be four load cells, one in each corner. The device may alternatively include eight load cells where there are four load cells positioned to be under each foot. This configuration may provide separate motion information for each foot.

In some examples, there may be one or more output mechanisms provided by the device. The feedback may be visual, audible or tactile. For example, the user's weight and blood pressure may be displayed on the visual display of the device. The measurements may alternatively or additionally be conveyed using audio (e.g., verbal) feedback. For example, a speaker may be provided in the device to indicate to the user a blood pressure value. In some examples, the device may also provide tactile or haptic feedback (e.g., via a vibration mechanism). The haptic feedback may indicate the beginning or end of a measurement. The haptic feedback may also convey information about the measurement. For example, a double tap vibration may indicate a good measurement (e.g., a measurement falling within healthy thresholds).

Although the disclosed device has been described in examples in which the user stands on the device, in some examples the device may be used with the user in a seated position. For example, the user may sit on a chair and place his or her feet flat on the device. The obtained ECG and IPG signals will remain very similar to those obtained in a standing position. The obtained BCG signal will remain detectable in a seated position, but may have a substantially different morphology. Due to changes in the signals, a separate calibration procedure may be required while seated. In some examples, the user may provide input to indicate to the device that the user is in a seated position. In some examples, the device may determine that the user is in a seated position, for example during a calibration phase or by detecting that the measured weight is less than expected. In some examples, the device may reference different predefined thresholds for detecting a possible adverse event if the user is using the device in a seated position. The device may store separate sets of baseline information for a user for the standing and seated positions.

In some examples, the device may be in the form of a chair. Sensors may be incorporated into the chair, where the force sensor(s) may be positioned in the legs of the chair, to detect the force exerted by the user on the chair, and the electrical signal sensors may be positioned on the arm rests and/or back of the chair. For example, the electrical signal sensors may be positioned to be touched by the hands of the user, alternatively they may be capacitive sensors positioned to obtain signals from the user through the user's clothing. In other example variations, a similar configuration of sensors may be used for the device in the form of a bed or other such object.

In some examples, the device may be similar to a bathroom scale, with a handlebar or support bar attachment. Force sensor(s) may be positioned in the platform of the scale, while electrical signal sensors may be positioned on the handlebar or support bar, to be gripped by the user's hands. This configuration may be suitable for elderly users or those suffering from difficulties in balancing or standing for the duration of a measurement, for example.

In some examples, the sensors may be directly embedded into a floor or may be integrated into a surface, such as a tile, that makes up part of a floor. The user may stand on the floor or surface, over the sensors, to obtain his/her cardiovascular information. Such a configuration may make the process of obtaining cardiovascular metrics even less intrusive.

In various examples discussed herein, the disclosed device may enable various biological parameters to be measured relatively easily and conveniently. The combination of cardiac metrics with measurements of weight and body fat is critical to providing actionable health information. Understanding the correlation between weight and heart health is important to a patient and physician in the treatment of many diseases. Body weight is one of the dominant factors impacting hypertension. Consequently, lifestyle changes leading to weight loss are an extremely effective way to lower high blood pressure. The majority of heart disease may be attributed to preventable causes, such as poor diet and lack of exercise. By incorporating weight and blood pressure in one platform, a user can easily understand the impact of an unhealthy lifestyle not just on physical appearance, but also long term health. Beyond hypertension, weight and blood pressure may be critical measurements in the monitoring of other cardiovascular conditions. For example, diligent out-of-hospital monitoring of weight and blood pressure may be critical to heart failure management. One of the early signs of heart failure decompensation is rapid weight gain due to fluid build-up in the legs (a condition known as edema). By measuring this predictor of heart failure deterioration with other cardiovascular metrics, the disclosed device may be a suitable device for the home monitoring of heart failure patients. The disclosed device in a form similar to a bathroom scale augmented with cardiac sensors may not require any new habits from a user. Simply replacing an existing weight scale with an example of the disclosed device may allow a user to obtain comprehensive health metrics without the added complexity of additional devices. In some examples, the disclosed device may take a form similar to any bathroom scale, with the addition of the sensors to measure biological signals, to determine blood pressure and heart function.

At least some aspects disclosed may be embodied, at least in part, in software. That is, some disclosed techniques and methods may be carried out in a computer system or other data processing system in response to its processor, such as a microprocessor, executing sequences of instructions contained in a memory, such as ROM, volatile RAM, non-volatile memory, cache or a remote storage device.

A computer readable storage medium may be used to store software and data which when executed by a data processing system causes the system to perform various methods or techniques of the present disclosure. The executable software and data may be stored in various places including for example ROM, volatile RAM, non-volatile memory and/or cache. Portions of this software and/or data may be stored in any one of these storage devices.

Examples of computer-readable storage media may include, but are not limited to, recordable and non-recordable type media such as volatile and non-volatile memory devices, read only memory (ROM), random access memory (RAM), flash memory devices, floppy and other removable disks, magnetic disk storage media, optical storage media (e.g., compact discs (CDs), digital versatile disks (DVDs), etc.), among others. The instructions can be embodied in digital and analog communication links for electrical, optical, acoustical or other forms of propagated signals, such as carrier waves, infrared signals, digital signals, and the like. The storage medium may be the internet cloud, or a computer readable storage medium such as a disc.

Furthermore, at least some of the methods described herein may be capable of being distributed in a computer program product comprising a computer readable medium that bears computer usable instructions for execution by one or more processors, to perform aspects of the methods described. The medium may be provided in various forms such as, but not limited to, one or more diskettes, compact disks, tapes, chips, USB keys, external hard drives, wire-line transmissions, satellite transmissions, internet transmissions or downloads, magnetic and electronic storage media, digital and analog signals, and the like. The computer useable instructions may also be in various forms, including compiled and non-compiled code.

At least some of the elements of the systems described herein may be implemented by software, or a combination of software and hardware. Elements of the system that are implemented via software may be written in a high-level procedural language such as object oriented programming or a scripting language. Accordingly, the program code may be written in C, C++, J++, or any other suitable programming language and may comprise modules or classes, as is known to those skilled in object oriented programming. At least some of the elements of the system that are implemented via software may be written in assembly language, machine language or firmware as needed. In either case, the program code can be stored on storage media or on a computer readable medium that is readable by a general or special purpose programmable computing device having a processor, an operating system and the associated hardware and software that is necessary to implement the functionality of at least one of the embodiments described herein. The program code, when read by the computing device, configures the computing device to operate in a new, specific and predefined manner in order to perform at least one of the methods described herein.

The embodiments of the present disclosure described above are intended to be examples only. The present disclosure may be embodied in other specific forms. Alterations, modifications and variations to the disclosure may be made without departing from the intended scope of the present disclosure. While the systems, devices and processes disclosed and shown herein may comprise a specific number of elements/components, the systems, devices and assemblies could be modified to include additional or fewer of such elements/components. For example, while any of the elements/components disclosed may be referenced as being singular, the embodiments disclosed herein could be modified to include a plurality of such elements/components. Selected features from one or more of the above-described embodiments may be combined to create alternative embodiments not explicitly described. All values and sub-ranges within disclosed ranges are also disclosed. The subject matter described herein intends to cover and embrace all suitable changes in technology. All references mentioned are hereby incorporated by reference in their entirety.

1. A device for measuring a biological signal, the device comprising:
a device body for supporting a user;
one or more sensors for sensing at least one biological signal, the biological signal being a cardiovascular signal, the one or more sensors including at least one of:
at least one force sensor positioned to sense a force exerted on the device body, wherein the at least one force sensor measures one of the at least one biological signal as a varying force; or
at least two electrical signal sensors positioned to detect electrical signals from the user when the device is in use, wherein the at least two electrical signal sensors cooperate with each other to obtain one of the at least one biological signal from the user via the feet of the user; and
a processor housed in the device body, the processor coupled to the one or more sensors to receive the at least one biological signal;
wherein the processor is configured to:
determine, from the at least one biological signal, one or more biological parameters; and
generate output representative of the one or more biological parameters.

2. The device of claim 1, wherein the device body comprises a platform on which the feet of the user are placed when the device is in use.

3. A device for measuring a biological signal, the device comprising:
a device body for supporting a user;
sensors for sensing respective biological signals, the biological signals including a cardiovascular signal, the sensors including:
at least one force sensor positioned to sense a force exerted on the device body, wherein the at least one force sensor measures one biological signal as a varying force; and
at least two electrical signal sensors positioned to detect electrical signals from the user when the device is in use, wherein the at least two electrical signal sensors cooperate with each other to obtain another biological signal from the user; and
a processor housed in the device body, the processor coupled to the sensors to receive the biological signals;
wherein the processor is configured to:
determine, from the biological signals, one or more biological parameters; and
generate output representative of the one or more biological parameters.

4. The device of claim 3, wherein the device body comprises a platform for supporting the user when the device is in use and further comprises a bar on which the hands of the user are placed when the device is in use, wherein the at least two electrical signal sensors are positioned on the bar to detect electrical signals via each hand of the user.

5. The device of claim 3, wherein the device body comprises a chair with armrests, wherein the at least two electrical signal sensors are positioned on the armrests to detect electrical signals via each hand of the user.

6. The device of claim 1, wherein the biological signal includes an electrocardiograph (ECG) signal measured as a potential difference between the at least two electrical signal sensors.

7. The device of claim 1, wherein the biological signal includes an impedance plethysmograph (IPG) signal measured as a varying voltage between the at least two electrical signal sensors.

8. The device of claim 1, wherein the biological signal includes a ballistocardiograph (BCG) signal measured by the at least one force sensor as a varying force.

9. The device of claim 1, wherein the at least two electrical signal sensors are contact electrodes or capacitive sensors.

10. (canceled)

11. The device of claim 1, wherein the processor receives at least two biological signals, wherein the processor is further configured to:
select one biological signal as a gating signal; and
process the received biological signals using the gating signal as a reference.

12. The device of claim 1, wherein the processor is further configured to:
calculate an ensemble average of the biological signal over a plurality of samples; and
determine the one or more biological parameters on the basis of the ensemble average.

13. The device of claim 1, wherein the processor is further configured to:
compare at least one of the biological signal or the one or more biological parameters with at least one of a preset threshold or a baseline value;
determine, on the basis of the comparison, whether the at least one of the biological signal or the one or more biological parameters is indicative of a possible or expected adverse event; and
generate output representative of the possible or expected adverse event.

14-16. (canceled)

17. The device of claim 1, further comprising a communication interface for at least one of receiving input from an external system;
wherein the processor is further configured to:
receive information about one or more user characteristics from the external system; and
determine the one or more biological parameters in accordance with the one or more user characteristics.

18. The device of claim 1, wherein the processor is further configured to:
receive information about one or more measurements related to the user's body;
use the received information to perform calibration calculations on the cardiovascular signal; and
determine the one or more biological parameters based on the calibrated cardiovascular signal.

19. (canceled)

20. The device of claim 1, wherein the processor is further configured to:
receive information about one or more measurements related to the user's body; and
use the received information to calculate a cardiovascular metric.

21. (canceled)

22. The device of claim 1, wherein the at least two electrical signal sensors are configured to detect electrical signals from the user without direct contact with the user's skin.

23-24. (canceled)

25. The device of claim 24, wherein the one or more biological parameters comprise a blood pressure, and wherein the processor is further configured to determine the blood pressure by:
determining at least two independent systolic time intervals (STIs) from the biological signal; and
using each of the at least two STIs to independently calculate systolic and diastolic blood pressures.

26. The device of claim 1, further comprising a plurality of force sensors positioned to sense a force exerted on the device body by the user, wherein the processor is further configured to:
determine, from measurements by the force sensors, a center of pressure exerted by the user on the device body;
determine any variance or deviation in the center of pressure over a measurement duration;
when the variance or deviation exceeds a predetermined threshold, determine that the user is exhibiting dizziness or poor balance; and
generate output based on the determination that the user is exhibiting dizziness or poor balance.

27. The device of claim 26, wherein the output based on the determination that the user is exhibiting dizziness or poor balance is output indicating possible low blood pressure or possible side effect of a medication.

28. A server for accessing biological information, the server comprising:
a processor for communication with the device of claim 1;
the processor being configured to provide the device with access to a database of stored biological information, the database including biological information obtained using the device; and
the processor being configured to provide an online portal for accessing information stored in the database by an external system.

29-30. (canceled)
